(19) United States
(12) Patent Application Publication
(10) Pub. No.: US 2025/0107707 A1
(43) Pub. Date: Apr. 3, 2025

(54) VCSEL ARRAYS FOR OCT IMAGING

(71) Applicant: ACUCELA INC., Seattle, WA (US)

(72) Inventors: Ryo KUBOTA, Seattle, WA (US); Amitava GUPTA, Seattle, WA (US)

(73) Assignee: ACUCELA INC., Seattle, WA (US)

(21) Appl. No.: 18/834,536

(22) PCT Filed: Feb. 17, 2023
(86) PCT No.: PCT/US2023/062794

§ 371 Date: Jul. 30, 2024

Related U.S. Application Data
(60) Provisional application No. 63/268,254, filed on Feb. 18, 2022.

(51) Int. Cl. A61B 3/10; G02B 26/08

(52) U.S. Cl. CPC A61B 3/102; G02B 26/0825

(57) ABSTRACT

A swept source OCT system comprises plurality of light beams from an array of VCSELs. The plurality of light beams is imaged onto a retina of an eye and the image of the plurality of light beams formed on the retina is imaged onto a detector array. In some embodiments, a portion of the light from the plurality of light beams is reflected from a reference mirror, combined with the plurality of light beams returned from the retina, and imaged onto the detector array to generate an interference signal. The wavelength of the plurality of light beams is swept to generate a swept source signal on the detector and recorded to generate an interference signal for each of the plurality of light beams. The interference signal can be processed to generate a reflectance signal for each of the plurality of light sources.

### RELATED APPLICATIONS

This application claims the benefit under 35 U.S.C. § 119 (e) of U.S. Provisional Patent Application No. 63/268,254, filed Feb. 18, 2022, which is incorporated, in its entirety, by this reference.

The subject matter of the present application is related to PCT/US2019/038270, filed on Jun. 20, 2019, entitled “MINIATURIZED MOBILE, LOW COST OPTICAL COHERENCE TOMOGRAPHY SYSTEM FOR HOME BASED OPHTHALMIC APPLICATIONS”, published as WO/2019/246412 on Dec. 26, 2019, and U.S. application Ser. No. 17/647,585, filed on Jan. 10, 2022, now U.S. Pat. No. 11,497,396, issued Nov. 15, 2022, entitled “AXIAL LENGTH MASUREMENT MONITOR”, the entire disclosures of which are incorporated herein by reference.

### BACKGROUND

Optical coherence tomography (OCT) has been used in many fields to provide high resolution tomographic images of tissue and other structures. One field where OCT is often used is ophthalmic examinations of tissues such as the cornea, lens, and retina. For example, it can be helpful to monitor retinal thickness to diagnose and monitor the onset of retinal diseases. OCT imaging of the ocular lens can be helpful to diagnose and treat cataracts, and OCT imaging to determine the axial length of the eye can be helpful for monitoring the progression of myopia, for example. Although widely used in clinical applications, OCT generally has not been used for in home monitoring in at least some instances. In home monitoring would be beneficial because it could lead to detection of diseases sooner and improved monitoring and treatment.

Although useful, the prior approaches to OCT imaging can be less than ideal in at least some respects. Work in relation to the present disclosure suggests that at least some of the approaches can be more complex and have more moving parts than would be ideal. Also, the prior approaches are not well suited for in home monitoring. Also, while the prior approaches to OCT imaging can provide very good axial resolution along the path of the measurement beam, the lateral resolution can be less than ideal in at least some respect. Although a scanning mirror can be used to scan the measurement beam, this can lead to less spatial resolution lateral to the beam than would be ideal.

In light of the above, it would be helpful to have improved systems and methods for OCT imaging that overcome at least some of the aforementioned limitations.

### SUMMARY

Embodiments of the present disclosure provide improved systems and methods for OCT imaging. The presently disclosed systems and methods allow for the substantially simultaneous generation of A-scan measurements from a plurality of swept source light beams, which can increase the spatial resolution and, in some embodiments, decease the acquisition time to image an area. In some embodiments, the area can be measured without a movable mirror to can the measurement beam, which can decrease the complexity of the system and provide imaging with fewer moving parts. Alternatively, a scanning mirror can be used to displace the area measured with the plurality of beams and provide increased spatial resolution with the plurality of swept source light beams. The plurality of light beams can be generated in many ways, and in some embodiments, the plurality of light beams is generated with an array of vertical cavity surface emitting lasers (VCSELs).

In some embodiments, a plurality of light beams from a plurality of light sources such as a plurality of VCSELs is imaged onto the retina of the eye and the image of the plurality of light beams formed on the retina is imaged onto a detector array. In some embodiments, a portion of the light from the plurality of light beams is reflected from a reference mirror, combined with the plurality of light beams returned from the retina, and imaged onto the detector array to generate an interference signal. The wavelength of the plurality of light beams is swept to generate a swept source signal on the detector and recorded to generate an interference signal for each of the plurality of light beams. The interference signal can be processed to generate a reflectance signal for each of the plurality of light sources.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1A shows a simplified diagram of the human eye;

FIG. 1B shows a perspective view of a monocular optical coherence tomography (OCT) system for measuring eyes of a user, in accordance with some embodiments;

FIG. 2 shows a schematic of a system allowing a patient to measure axial length at multiple time points and to communicate the results, in accordance with some embodiments;

FIG. 3A shows a handheld optical coherence tomography system utilizing Bluetooth communication, in accordance with some embodiments;

FIG. 3B shows a handheld OCT system utilizing the Global System for Mobile Communications (GSM), in accordance with some embodiments;

FIG. 4 shows a VCSEL array imaged into the eye with an OCT system, in accordance with some embodiments;

FIG. 5 shows the system of FIG. 4 in which the one or more lenses comprises a lenslet array to form a plurality of images of the retina, in accordance with some embodiments;

FIG. 6 shows a swept source VCSEL configured to generate a plurality of light sources with a lenslet array and a lens, in accordance with some embodiments;

FIG. 7 shows an etalon optically coupled to one or more lenses to generate a wavelength signal, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

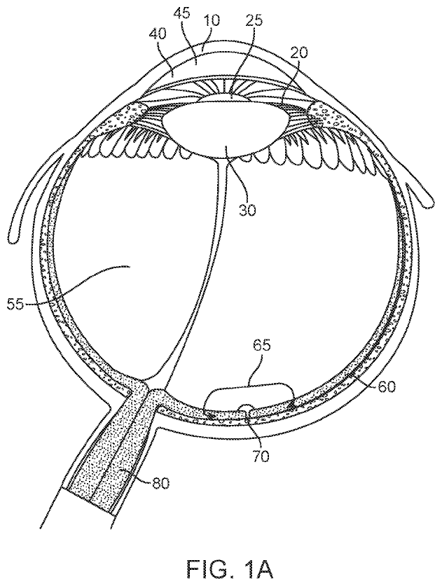

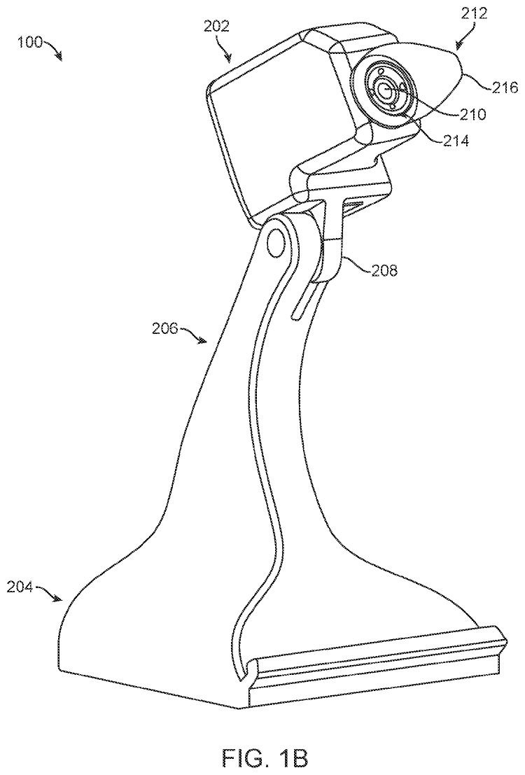

FIG. 1A shows a simplified diagram of the human eye. Light enters the eye through the cornea 10. The iris 20 controls the amount of light allowed to pass by varying the size of the pupil 25 that allows light to proceed to the lens 30. The anterior chamber 40 contains aqueous humor 45 which determines the intraocular pressure (IOP). The lens 30 focuses light for imaging. The focal properties of the lens are controlled by muscles which reshape the lens. Focused light passes through the vitreous chamber, which is filled with vitreous humor 55. The vitreous humor maintains the overall shape and structure of the eye. Light then falls upon the retina 60, which has photosensitive regions. In particular, the macula 65 is the area of the retina responsible for receiving light in the center of the visual plane. Within the macula, the fovea 70 is the area of the retina most sensitive to light. Light falling on the retina generates electrical signals which are passed to the optic nerve 80 and then to the brain for processing.

Several disorders give rise to reduced optical performance of the eye. In some cases, the axial length of the eye is undesirably long, which is related to myopia of the patient. In some cases, the intraocular pressure (IOP) is either too high or too low. This is caused, for instance, by too high or too low of a production rate of aqueous humor in the anterior chamber or drainage of aqueous humor from the anterior chamber, for example. In other cases, the retina is too thin or too thick. This arises, for instance, due to the buildup of fluid in the retina. Diseases related to an abnormal retinal thickness (RT) include glaucoma, macular degeneration, diabetic retinopathy, macular edema and diabetic macular edema, for example. In some cases, a healthy range of RT is from 175 μm thick to 225 μm thick. In general, abnormalities in either the IOP or the RT or both are indicative of the possible presence of one of several ophthalmological diseases. Additionally, the IOP or the RT vary in response to ophthalmological treatments or other procedures. Therefore, it is desirable to have a means to measure the IOP and/or RT for diagnosis of ophthalmological diseases and to assess the effectiveness of treatments for a given patient. In some cases, it is desirable to measure the thickness of one or more retinal layers, for example the thickness of a plurality of layers. In addition, it is desirable to process data obtained from an OCT system to assist in identifying fluid pockets or regions in the eye, as these may indicate a change in eye health.

FIG. 1B shows a perspective view of a monocular optical coherence tomography (OCT) system 100 comprising an array of VCSELs for measuring eyes of a user. The OCT system 100 includes a head 202, a base 204, and a neck 206 therebetween. The head 202 is connected to the neck 206 by a coupling 208 that allows articulation of the head 202 in some embodiments. The head may be covered with a housing that encloses optical modules, scanning modules, and other related circuitry and modules to allow the OCT system 100 to measure eyes of a user, one eye at a time.

In some embodiments, the head 202 further includes a lens 210, and eyecup 212, and one or more LED lights 214. The lens 210 may be configured to direct one or more light sources from within the head 202 to focus on the retina of an eye. The eyecup 212 may be configured to locate the head of a patient, and thereby locate an eye of a patient for scanning and testing. The eyecup 212 may be rotatable, so that a protruding portion 216 may be located adjacent to an eye of a patient and extend along the side of the head (e.g., adjacent the patient's temple) when the patient's head is properly oriented to the OCT system 100. The eyecup 212 may be coupled to a sensor configured to detect the rotational orientation of the eyecup 212. In some embodiments, the OCT system 100 is configured to detect the rotational orientation of the eyecup 212 and thereby determine whether the patient has presented her right eye or left eye for scanning and measuring. More particularly, in some embodiments, the protruding portion 216 of the eyecup 212 may extend to be adjacent to either the right temple or the left temple of a patient, and thereby determine which eye of the patient is being measured. In some embodiments, eyecup 212 comprises a patient support. The patient support may comprise a headrest or a chinrest, either alternatively or in combination with the eyecup 212.

In some embodiments, a coupling 208 connects the head 202 to the neck 206 and allows a pivotal movement about the coupling. The coupling 208 may be any suitable coupling, which may be rigid, articulating, rotational, or pivotal according to embodiments. In some instances, the coupling includes a threaded fastener and a threaded nut to tighten the head against the neck in a desired orientation. The threaded nut may be operable by hand, and may comprise a knurled knob, a wing nut, a star nut, or some other type of manually operated tightening mechanism. The coupling may alternatively or additionally comprise any suitable member that allows adjustment of the angle of the head relative to the neck, and may include a cam, a lever, or a detent, and may alternatively or additionally include friction increasing structures, such as roughened surfaces, peaks and valleys, surface textures, and the like.

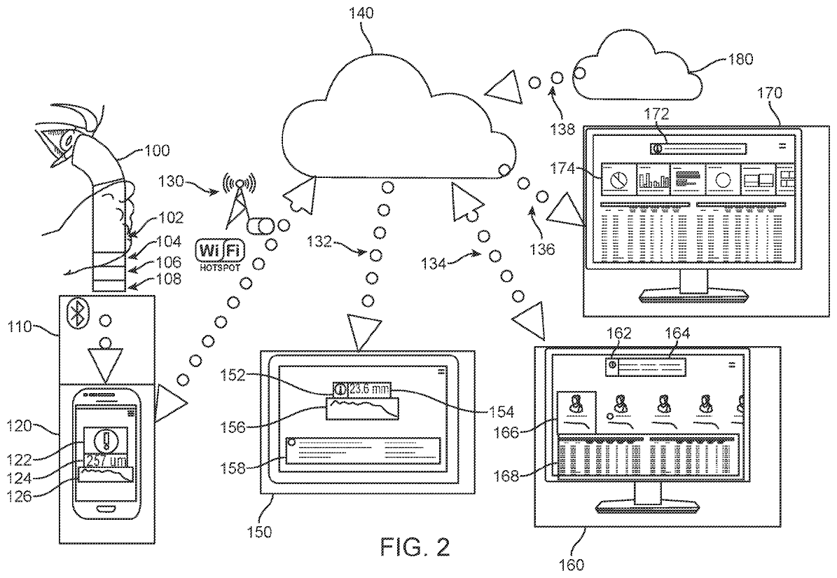

FIG. 2 shows a schematic of a system allowing a patient to measure the eye with an OCT array at multiple time points and to communicate the results. The patient looks into a handheld OCT system 100 to obtain a measurement of the AL. In some embodiments, the handheld OCT system comprises optics 102, electronics 104 to control and communicate with the optics, a battery 106, and a transmitter 108. In some instances, the transmitter is a wired transmitter. In some cases, the transmitter is a wireless transmitter. In some cases, the handheld OCT system 100 communicates the results via a wireless communication channel 110 to a mobile patient system 120 such as the patient's smartphone or other portable electronic system. In some cases, the wireless communication is via Bluetooth communication. In some embodiments, the wireless communication is via Wi-Fi communication. In other embodiments, the wireless communication is via any other wireless communication known to one having skill in the art. Although reference is made to wireless communication, in some embodiments the OCT system connects by wired communication to the patient mobile system and the patient mobile system connects wirelessly to a remote server such as a cloud-based server.

In some cases, the results are fully processed measurements of the OCT images of the eye. In some cases, all processing of the OCT data is performed on the handheld OCT system. For instance, in some embodiments, the handheld OCT system includes hardware or software elements that allow the OCT optical waveforms to be converted into electronic representations. In some cases, the handheld OCT system further includes hardware or software elements that allow processing of the electronic representations to extract, for instance, a measurement of the axial length or retinal thickness, for example.

In some cases, the results are electronic representations of the raw optical waveforms obtained from the OCT measurement. For instance, in some embodiments, the handheld OCT system includes hardware or software elements that allow the OCT optical waveforms to be converted into electronic representations. In some cases, these electronic representations are then passed to the mobile patient system for further processing to extract, for instance, a measurement of the RT.

In some cases, the patient receives results and analysis of the OCT measurement on the patient mobile app. In some embodiments, the results include an alert 122 alerting the patient that the results of the measurement fall outside of a normal or healthy range. In some cases, the results also include a display of the measured value 124. For instance, in some cases a measurement of the retina or axial length produces a result with a specific value in millimeters (“mm”), e.g. 23.6 mm. In some instances, this result corresponds to a change in length outside a desired range. This causes the system to produce an alert and to display the measured value on the patient mobile app. In some embodiments, the alert is transmitted to a healthcare provider, such as a treating physician. In some embodiments, the results also include a chart 126 showing a history of the patient's axial length and retinal thickness measurements over multiple points in time.

In some instances, the patient mobile system communicates the results of the measurement via a communication means 130 to a cloud-based or other network-based storage and communications system 140. In some embodiments, the communication means is a wired communication means. In some embodiments, the communication means is a wireless communication means. In some cases, the wireless communication is via Wi-Fi communication. In other cases, the wireless communication is via a cellular network. In still other cases, the wireless communication is via any other wireless communication known to one having skill in the art. In specific embodiments, the wireless communication means is configured to allow transmission to or reception from the cloud-based or other network-based storage and communications system.

Once stored in the cloud, the results are then transmitted to other systems, in specific embodiments. In some cases, the results are transmitted via a first communication channel 132 to a patient system 150 on the patient's computer, tablet, or other electronic system. In some embodiments, the results are transmitted via a second communication channel 134 to a physician system 160 on the patient's physician's computer, tablet, or other electronic system. In some instances, the results are transmitted via a third communication channel 136 to an analytics system 170 on another user's computer, tablet, or other electronic system. In some embodiments, the results are transmitted via a fourth communication channel 138 to a patient administration system or hospital administration system 180. In some cases, each of the systems has appropriate software instructions to perform the associated function(s) as described herein.

In specific embodiments, the first communication channel is a wired communication channel or a wireless communication channel. In some cases, the communication is via Ethernet. In other cases, the communication is via a local area network (LAN) or wide area network (WAN). In still other cases, the communication is via Wi-Fi. In yet other cases, the communication is via any other wired or wireless communication channel or method known to one having skill in the art. In some embodiments, the first communication channel is configured to allow transmission to or reception from the cloud-based or other network-based storage and communications system. In some cases, the first communication channel is configured to only allow reception from the cloud-based or other network-based storage and communications system.

In some cases, the second communication channel is a wired communication channel or a wireless communication channel. In some instances, the communication is via Ethernet. In specific embodiments, the communication is via a local area network (LAN) or wide area network (WAN). In other embodiments, the communication is via Wi-Fi. In still other embodiments, the communication is via any other wired or wireless communication channel or method known to one having skill in the art. In some cases, the second communication channel is configured to allow transmission to or reception from the cloud-based or other network-based storage and communications system. In some embodiments, the second communication channel is configured to only allow reception from the cloud-based or other network-based storage and communications system.

In specific cases, the third communication channel is a wired communication channel or a wireless communication channel. In some instances, the communication is via Ethernet. In other instances, the communication is via a local area network (LAN) or wide area network (WAN). In still other instances, the communication is via Wi-Fi. In yet other instances, the communication is via any other wired or wireless communication channel or method known to one having skill in the art. In some embodiments, the third communication channel is configured to allow transmission to or reception from the cloud-based or other network-based storage and communications system. In some cases, the third communication channel is configured to only allow reception from the cloud-based or other network-based storage and communications system.

In some embodiments, the fourth communication channel is a wired communication channel or a wireless communication channel. In some cases, the communication is via Ethernet. In other cases, the communication is via a local area network (LAN) or wide area network (WAN). In still other cases, the communication is via Wi-Fi. In yet other cases, the communication is any other wired or wireless communication channel or method known to one having skill in the art. In some instances, the fourth communication channel is configured to allow transmission to or reception from the cloud-based or other network-based storage and communications system. In other cases, the fourth communication channel is configured to only allow reception from the cloud-based or other network-based storage and communications system.

In some embodiments, the patient's physician receives the results and analysis of the OCT measurements on the physician system 160. In some instances, the results include an alert 162 alerting the physician that the results of the measurement correspond to a potentially significant change from baseline. In some cases, the results also include an alert 164 informing the physician of the patient's measurement. In some embodiments, the alert includes a suggestion that the physician call the patient to schedule an appointment or to provide medical assistance. In some embodiments, the results also include a display 166 showing the most recent measurements and historical measurements for each of the physician's patients. In specific cases, the physician system also displays contact and historical information 168 for each of the physician's patients.

In some embodiments, the other user receives results and analysis of the OCT measurement on the analytics system 170. In some instances, the other user is a researcher investigating the efficacy of a new form of treatment. In other cases, the other user is an auditor monitoring the outcomes of a particular physician or care facility. To protect the patient's privacy, in some cases the analytics system is restricted to receive only a subset of a given patient's information. For instance, the subset is restricted so as not to include any personally identifying information about a given patient. In some cases, the results include an alert 172 alerting or indicating that a large number of abnormal or undesirable measurements have been obtained in a specific period of time. In some cases, the results include one or more graphical representations 174 of the measurements across a population of patients.

In some embodiments, the patient's clinical, hospital, or other health provider receives results and analysis of the AL measurement on the patient administration system or hospital administration system 180. In some cases, this system contains the patient's electronic medical record. In some cases, the results and analysis provide the patient's health provider with data allowing the provider to update the treatment plan for the patient. In some instances, the results and analysis allow the provider to decide to call the patient in for an early office visit. In some instances, the results and analysis allow the provider to decide to postpone an office visit.

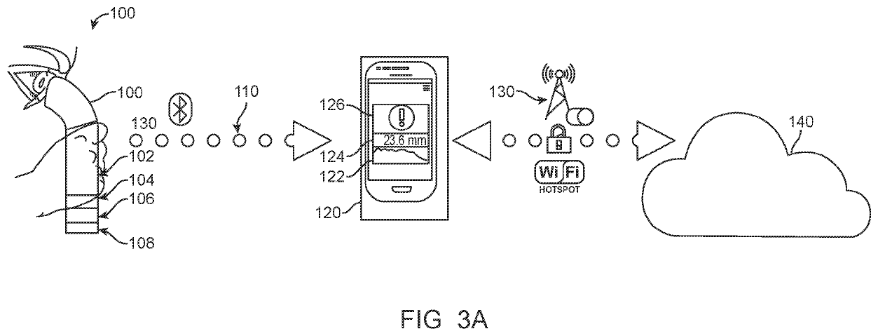

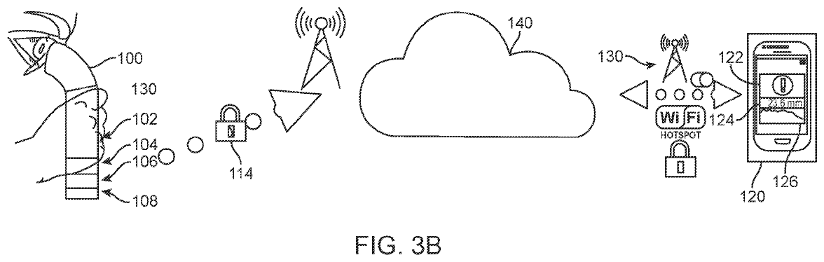

FIG. 3A shows a handheld OCT system comprising an array of VCSELs utilizing short-range wireless communication, in accordance with some embodiments. In some embodiments, the handheld OCT system 100 comprises optics 102, electronics to control and communicate with the optics 104, a battery 106, and a wireless transmitter 108. In some cases, the wireless transmitter is a Bluetooth transmitter. In some cases, the communication is via a Bluetooth wireless communication channel 110. In some instances, the handheld OCT system communicates the results via the Bluetooth channel to a mobile patient system 120 on the patient's smartphone or other portable electronic system.

In some instances, the results include an alert 122 alerting the patient that the results of the measurement fall outside of a desired range. In specific embodiments, the results also include a display of the measured value 124. In specific embodiments, the results also include a chart 126 showing a history of the patient's OCT measurements over multiple points in time.

In some cases, the patient mobile system application communicates the results of the measurement via a wireless communication means 130 to a cloud-based or other network-based storage and communications system 140.

FIG. 3B shows a handheld OCT system comprising an array of VCSELs capable of communicating directly with a cloud-based storage and communication system without reliance on a user system such as a smartphone, in accordance with some embodiments. In some embodiments, the handheld OCT system 100 comprises optics 102, electronics to control and communicate with the optics 104, a battery 106, and a wireless transmitter 108. In some cases, the wireless transmitter is a GSM transmitter. In some instances, the results from one or more AL measurements are stored on the handheld OCT system. In some cases, the GSM transmitter establishes wireless communication with a cloud-based or other network-based storage and communications system 140 via a wireless communication channel 114. In specific cases, the wireless communication is via a GSM wireless communication channel. In other embodiments, the system utilizes third generation (3G) or fourth generation (4G) mobile communications standards. In such cases, the wireless communication is via a 3G or 4G communication channel.

In specific embodiments, the patient mobile system 120 receives the results of the measurement via a wireless communication means 130 from the cloud-based or other network-based storage and communications system 140.

Once obtained from the cloud-based or other network-based storage and communications system, the results of the OCT measurements are viewed in the patient mobile application, in some instances. In some cases, the results include an alert 122 alerting the patient that the results of the measurement fall outside of a normal or healthy range. In some instances, the results also include a display of the measured value 124.

In some cases, the handheld OCT system of FIGS. 3A and 3B is configured to be held in close proximity to the eye. For instance, in specific embodiments, the system is configured to be held in front of the eye with the detector and VCSEL array at a distance of no more than 200 mm from the eye. In other embodiments, the systems are configured to be held in front of the eye with the detector at a distance of no more than 150 mm, no more than 100 mm, or no more than 50 mm from the eye. In specific instances, the handheld OCT systems further comprise housing to support the light source, optical elements, detector, and circuitry. In some cases, the housing is configured to be held in a hand of a user. In some cases, the user holds the systems in front of the eye to direct the light beam into the eye. In some instances, the systems include a sensor to measure which eye is being measured. For instance, in specific embodiments, the systems include an accelerometer or gyroscope to determine which eye is measured in response to an orientation of the housing. The systems optionally include an occlusion structure coupled to the housing and the sensor that determines which eye is measured. The occlusion structure occludes one eye while the other eye is measured. In some cases, the systems include a viewing target to align the light beams with a portion of the retina. For instance, in specific embodiments, the systems include a viewing target to align the light beams with a fovea of the eye. In some cases, the viewing target is a light beam. In some cases, the viewing target is a light emitting diode. In other cases, the viewing target is a vertical cavity surface emitting laser (VCSEL). In still further cases, the viewing target is any suitable viewing target as will be known to one having ordinary skill in the art.

In some embodiments, the handheld OCT systems of FIGS. 3A and 3B are small enough and light enough to be easily manipulated with one hand by a user.

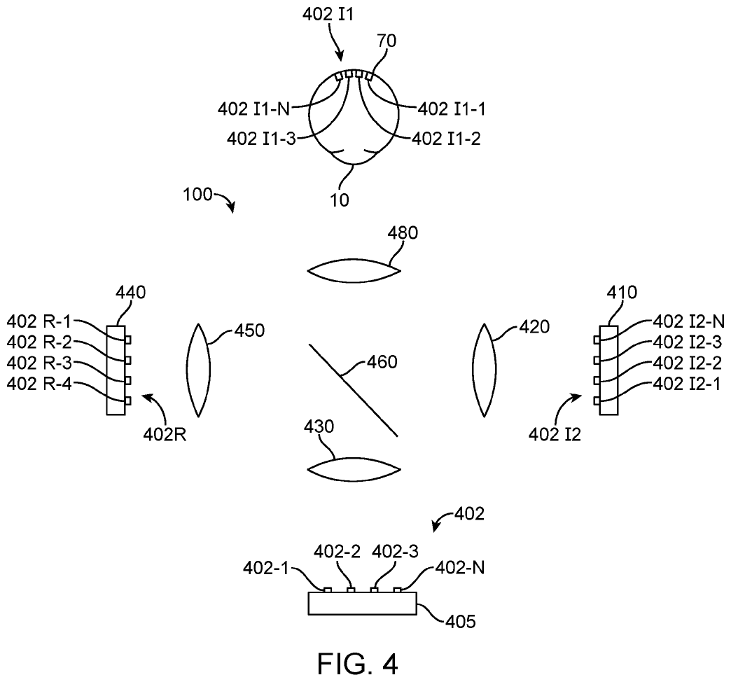

FIG. 4 shows a system 100 comprising the VCSEL array 405 imaged into the eye with one or more lenses 430, and light received from the eye imaged onto a detector array with one or more lenses after being combined with a reference beam, for example with a Mach-Zender configuration, to provide a plurality of OCT interferograms at a plurality of locations of the eye. The VCSEL array 405 comprises a plurality of light sources 402. The plurality of light sources 402 comprises a first light source 402-1, a second light source 402-2, a third light source 402-3 and an Nth light source 402-N. In some embodiments, the array of light source comprises a two-dimensional array of light sources.

Light from the VCSEL array 405 is directed toward one or more lenses 430, which may substantially collimate light from the plurality of VCSELs. The light from one or more lenses 430 is directed toward a beam splitter 460. The beam splitter 460 reflects a portion of the light along a reference optical path and transmits a second portion of the light along a measurement optical path. The beam splitter 460 may comprise any suitable beam splitter such as a partially reflective mirror or a polarizing beam splitter, for example. A first portion of the light from the beam splitter 460 is directed toward a mirror 440 along a reference optical path. Light reflected from mirror 440 is directed toward beam splitter 440 and transmitted through the beam splitter 460 toward detector 410. In some embodiments, one or more lenses 450 is located between the beam splitter 460 and mirror 440. In some embodiments, an additional and optional mirror is added to scan the image of plurality of light beams along the retina, such as a mirror coupled to a galvanometer, which can be used in combination with the plurality of light beams as described herein.

The detector 410 may comprise any suitable detector. In some embodiments, detector 410 comprises an array detector such as a complementary metal oxide semiconductor (CMOS) array or a charge coupled system (CCD) array, for example. In some embodiments, detector 410 comprises a plurality of pixels, in which each pixels comprises a width within a range from about 0.5 micrometers (um) to about 5 um, for example within a range from about 1 um to about 3 um. In some embodiments, the detector 410 comprise a clock speed of at least about 1 MHz.

The detector 410 may comprise any suitable photo sensing elements, such is silicon photo sensing elements. The plurality of photo sensing elements can be arranged in any suitable manner, such as with a rectangular array. In some embodiments, the detector 410 comprises a plurality of photo sensing elements at a plurality of locations corresponding to locations of the VCSELs imaged onto the retina and then onto the sensing elements, for example. In some embodiments, detector 410 comprises an ASIC, in which each of the plurality of photo sensing elements comprises a location corresponding to a location of a VCSEL imaged onto detector 410.

In some embodiments, the detector 410 is configured to sample at least about 100 A-scan measurements in parallel, for example. The number of A-scan measurements acquired substantially simultaneously, e.g. in parallel, can be related to the number of VCSELs of the array, and can be any suitable number of parallel A-scan measurements, such as at least about 1000 A-scan measurements, at least about 10,000 A-scan measurements, or at least about 100,000 A-scan measurements for example. The number of VCSELs of the array may comprise any suitable number, such as at least about 100 VCSELs, at least about 1000 VCSELs, at least about 10,000 VCSELs or at least about 100,000 VCSELs. The VCSELs can be arranged in any suitable manner, such as a 10×10 array, a 30×30 array, 64×64 array, a 128×128 array, a 256×256 array, a 300×300 array, or a 512×512 array, for example. The VCSELs of the array can be configured to sweep at a rate to provide the detector 410 sufficient time to capture the intensity profile over time for each VCSEL element for the full sweep over the wavelength range, such as a sweep time over the full wavelength range within a range from about 0.1 seconds to about 1 second, although the sweep time can be faster, for example.

In some embodiments detector 410 comprises an application specific integrated circuit (ASIC). Alternatively or in combination detector 410 comprises a CMOS detector array coupled to an ASIC for signal acquisition.

The portion of light transmitted through beam splitter 460 is directed along the measurement optical path toward lens 480 and the eye. The lens 480 may comprise one or more of a variable focus lens, a multifocal lens, or a bifocal lens, for example, in order to compensate for refractive error of the eye. The light transmitted through mirror is imaged inside the eye to form an image 402I1 of the light sources from the VCSEL array 405 within the eye with the optical power of the eye and the lens 480. In some embodiments, the image 402I1 formed inside the eye comprises a plurality of corresponding first images of the individual VCSEL light sources of the VCSEL array, for example an image 402I1-1 corresponding to light source 402-1, an image 402I1-2 corresponding to light source 402-2, image 402I1-3 corresponding to light source 402-3, and an image 402I1-N corresponding to light source 402-N.

Light returned from the retina is transmitted through lens 480 and reflected from mirror 460 toward one or more lenses 420 and detector 410. The light reflected from mirror 460 is transmitted through one or more lenses 420 to form an image 402I2 of the light returned from the inside of the eye on the detector array 410. In some embodiments, the image 405I1 of the VCSEL array 405 formed inside the eye is imaged onto the detector array 410 with one or more lenses 420 to form image 402I2. In some embodiments, the components of image 402I2 correspond to components of image 405I1 and the plurality of lights sources 402 of VCSEL array 405, e.g. the VCSELs of the VCSEL array 405. In some embodiments, the image 402I2 comprises an image 402I2-1 corresponding to image 402I1-1 and light source 402-1, an image 402I2-2 corresponding to image 402I1-2 and light source 402-2, image 402I2-3 corresponding to image 402I1-3 and light source 402-3, and an image 402I2-N corresponding to image 402I1-N and light source 402-N.

As each of the VCSELs of the array varies the wavelength, the intensity of the corresponding image varies and is captured with detector 410. The one or more VCSELs can be configured in many ways to sweep the wavelength of light emitted from the one or more VCSELs. In some embodiments, the VCSELs generate light and sweep wavelengths substantially simultaneously. In some embodiments, each of the VCSELs is driven with a common voltage, and each VCSEL sweeps the output wavelength independently in response to the common voltage, e.g. based on individual heating and wavelength output characteristics. Alternatively or in combination, the VCSELs may be swept sequentially, for example with sequential activation of each of the plurality of VCSELs. In some embodiments, each of the plurality of VCSELs is independently powered from other VCSELs to provide independent wavelength sweeping.

Detector 410 is coupled to a processor and the intensity signal for the image from each VCSEL is captured and processed. In some embodiments, detector 410 generates a time varying signal from the image for each of the plurality of VCSELs, e.g. from each VCSEL of the array. In some embodiments, the time varying signal comprises a time varying interferogram that can be transformed with a suitable transform such as a Fourier transform to generate a reflectance signal from different depths, such as an A-scan. In some embodiments, the time varying signal is adjusted in response to the wavelength sweeping characteristics of each of the one or more VCSELs, prior to being transformed, in order to provide a time varying signal which is more appropriate for transformation. In some embodiments, an A-scan is generated for each of the plurality of light sources, such as an A-scan for each of the plurality of VCSELs. The plurality of A-scans can be combined to generate an image of the eye corresponding to location of each VCSEL imaged onto the retina.

In some embodiments, one or more lenses 450 focuses light onto mirror 440 to form an image of the plurality of light sources 402 from VCSEL array 405 on mirror 440. In some embodiments, the light is focused to an image 402R which comprises a plurality of reference beams which form as illumination spots on mirror 440. In some embodiments, the image 402R comprises an image 402R-1 corresponding to light source 402-1, an image 402R-2 corresponding to light source 402-2, image 402R-3 corresponding to light source 402-3, up to an image 402R-N corresponding to light source 402-N. Each of these images is imaged onto the detector array 410, such that the locations of the detector array comprise the corresponding reference images 402R and image 402I1 from the eye. In some embodiments, image 402I2-1 comprises an image of image 402I1-1 and an image of 402R-1 overlapping at a first location of array 410; image 402I2-2 comprises an image of image 402I1-2 and an image of 402R-2 overlapping at a second location of array 410; image 402I2-3 comprises an image of image 402I1-3 and an image of 402R-3 overlapping at a third location of array 410; and image 402I2-N comprises an image of image 402I1-N and an image of 402R-N overlapping at an Nth location of array 410.

In some embodiments, each of the reference light sources is combined with a corresponding image of a VCSEL on the retina and swept with the corresponding VCSEL to generate the corresponding image of the light source from the VCSEL on detector. For example, image 402I2-1 may comprise a combination of image 402I1-1 from the eye and image 402R-1 from reference mirror 440, such that image 402I2-1 is modulated while the VCSEL corresponding to light source 402-1 is swept. Image 402I2-2 may comprise a combination of image 402I1-2 from the eye and image 402R-2 from reference mirror 440, such that image 402I2-2 is modulated while the VCSEL corresponding to light source 402-2 is swept. Image 402I2-3 may comprise a combination of image 402I1-3 from the eye and image 402R-3 from reference mirror 440, such that image 402I2-3 is modulated while the VCSEL corresponding to light source 402-3 is swept. Image 402I2-N may comprise a combination of image 402I1-N from the eye and image 402R-N from reference mirror 440, such that image 402I2-N is modulated while the VCSEL corresponding to light source 402-N is swept. Each of the VCSEL light sources may be swept independently or synchronously, or combinations thereof, for example.

The mirror 440 can be configured in many ways in accordance with the present disclosure. In some embodiments, mirror 440 comprises a substantially flat reflective surface. Alternatively mirror 440 may comprise a deformable mirror to provide compensation to the optical path difference between the mirror surface and the surface of the retina. The deformable mirror may comprise any suitable deformable mirror such as a deformable membrane mirror or a segmented mirror with discrete displacement elements, in order to compensate for the optical path difference.

The VCSEL array 405 can be configured in many ways in accordance with the present disclosure. In some embodiments, the array comprises a plurality of VCSELs which are configured to sweep the wavelength emitted from each VCSEL with heating of the VCSEL. The number of VCSELs may comprise any suitable number (N) of VCSELs, and the number N can be from about 10 to about 10,000 VCSELs, optionally from about 100 to about 1000 VCSELs, for example. The VCSELs may be arranged in an array of rows and columns, for example, such as an I×J array, in which I corresponds to the number of rows and J corresponds to the number of columns. The number of columns I can be within a range from about 10 to about 100 (or more) and the number J can be within a range from about 10 to about 100, for example. The range of sweeping can be any suitable range, for example from about 5 nm to about 20 nm, e.g. from about 5 nm to about 10 nm. Examples of suitable VCSELs and associated signal processing are described in “MINIATURIZED MOBILE, LOW COST OPTICAL COHERENCE TOMOGRAPHY SYSTEM FOR HOME BASED OPHTHALMIC APPLICATIONS”, published as WO/2019/246412 on Dec. 26, 2019, and U.S. application Ser. No. 17/647,585, filed on Jan. 10, 2022, entitled “AXIAL LENGTH MASUREMENT MONITOR”, the entire disclosures of which have been previously incorporated by reference. The VCSEL array can be fabricated in many ways, for example with the VCSELs formed on a semi-conductor die. In some embodiments, the VCSEL array 405 comprises an Application Specific Integrated Circuit (ASIC).

In some embodiments, the array of VCSELs comprises a plurality of VCSELs in which a mirror such as a MEMs mirror moves to sweep the wavelength, and the range of sweeping can be any suitable range such as from about 10 nm to about 100 nm or more. The mirrors of the one or more VCSELs may be driven synchronously to provide substantially simultaneously wavelength sweeping, or independently to provide independent wavelength sweeping, and combinations thereof.

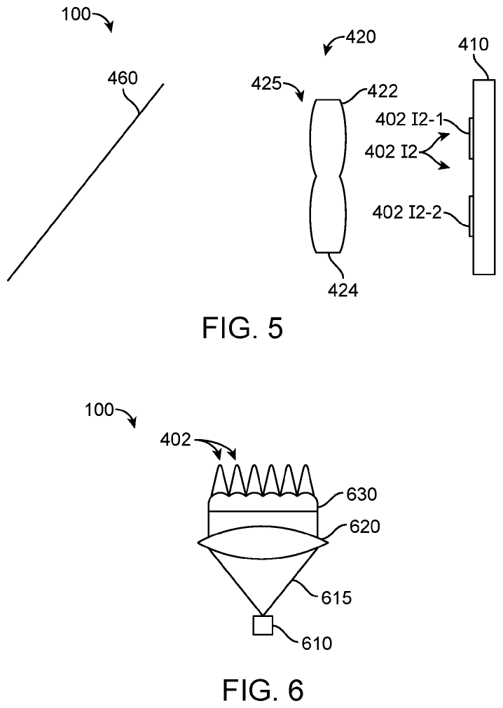

FIG. 5 shows the system of FIG. 4 comprising a lenslet array for form a plurality of images of the retina. In some embodiments, the one or more lenses 420 comprises a lenslet array 425. The lenslet array 425 comprises a plurality of lenses such as a first lens 422 and a second lens 424. In some embodiments, the second image 402I2 of the plurality of light sources from the VCSEL array comprises a first image 402I2-1 from a first lenslet and a second image 402I2-2 from a second lenslet. Each lens of the lenslet array forms an image of light from the retina and light from the reference mirror on the detector array, which can provide additional information with respect to the interference signal measured with the detector array 410. In some embodiments, the lenslet array forms a first image 402I2-1 of the light from the retina and a second image 402I2-2 on the detector array 410, up to an Nth image. Each of these images comprises the images of the light from the VCSEL elements that have been imaged onto the retina and reimaged onto the detector array 410, such as image 402I2-1, 402I2-2, 402I2-3, 402I2-N. This approach increases the number of images of the VCSEL array proportionally. For example, for a 10×10 VCSEL array comprising 100 VCSEL light sources and a 10×10 lenslet array comprising 100 lenses, the total number of images of light sources from the VCSELs is 10,000, which can provide additional information. For example, with a 100 lenslets, each VCSEL of the array is imaged 100 times simultaneously while the VCSEL is swept, which provides additional information to reconstruct the A-scan for the VCSEL at the location of the retina. In some embodiments, the number of lenses of the array multiplies the number of images of VCSEL elements by the number of lenslets, for example by 100 in the preceding embodiment. Although reference is made to each lenslet imaging the entire plurality of light sources from the VCSEL array, in some embodiments, each lenslet images a portion of the light from the VCSEL array.

The lenslet array may comprise any suitable number of lenses, such as within a range from about 4 to about 100 lenses. In some embodiments, the lenslet array comprises a two-dimensional array of lenslets, such as an M by N array of lenslets, in which M can be within a range from about 2 to about 10 lenses and N can be within a range from about 2 to about 10 lenses, and M and N can have the same value or different values, depending on the configuration. In some embodiments, the lenslet array comprises a 2×2 array, a 4×4 array, an 8×8 array, a 10×10 array, a 16×16 array or a 32×32 array, for example. The lenslet array may comprise any suitable lenslet array such as a gradient index (GRIN) lenslet array, a crossed cylindrical lenslet array, a diffractive lenslet array, or a holographic lenslet array, for example.

Although reference is made to a plurality of light sources 602 from a VCSEL array 405, the plurality of light sources can be generated by any suitable optical configuration.

The lenslet array 425 can be configured in many ways to image light from the retina. In some embodiments, the lenses of the lenslet array comprise a phased matched configuration, in which the lenslets are phase matched to each other.

FIG. 6 shows a swept source VCSEL 610 configured to generate a plurality of light sources 602 with a lenslet array 630 and a lens 620. The VCSEL 610 may comprise any suitable VCSEL as described herein. In some embodiments, light 615 from the VCSEL 610 is directed toward lens 620, which substantially collimates the light. The lenslet array 630 receives and focuses light 615 to generate the plurality of light sources 602, which can be incorporated into system 100 and light from the VCSEL swept as described herein.

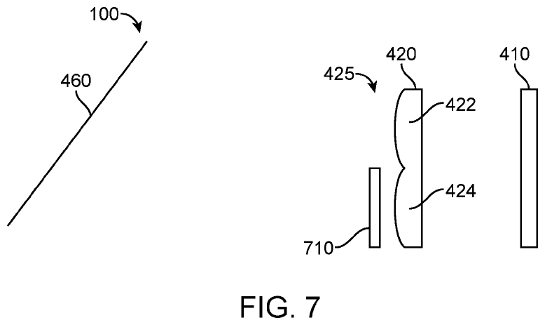

FIG. 7 shows one or more etalons 710 optically coupled to the one or more lenses 420 to generate a signal that varies with wavelength from the one or more VCSELS. In some embodiments, the one or more etalons 710 are configured to transmit wavelengths of light corresponding to constructive interference through the etalon and to decrease the transmission of light for wavelengths corresponding to destructive interference of light. As the VCSEL sweeps the output wavelength over a range of wavelengths, the etalon selectively transmits a first portion of the wavelengths and attenuates a second portion of the wavelengths, so as to generate a time varying intensity signal corresponding to the output wavelength. The time varying intensity signal can be used to adjust the reconstructed reflectance signal from the tissue layers. In some embodiments, the time varying intensity signal comprises a clock signal corresponding to the emission wavelength of each of the one or more VCSELs.

In some embodiments, the one or more lenses 420 comprises lenslet array 425 and the one or more etalons 710 comprise a plurality of etalons optically coupled to a portion of the plurality of lenslets in order to provide an etalon modulated signal to the detector array. In some embodiments each lenslet generates an image of the plurality of light beams reflected from the retina, and the intensity modulated signal through the etalon and a corresponding lenslet can be used to reconstruct the A-scan for a signal transmitted through a lenslet without the etalon. In some embodiments, a first lenslet 422 is not optically coupled to one or more etalons 710 and a second lenslet 424 is optically coupled to the one or more etalons 710 to generate the signal corresponding to wavelength at array detector 410, and both signals can be used to construct the reflectance signal.

As described herein, the computing systems and systems described and/or illustrated herein broadly represent any type or form of computing system or system capable of executing computer-readable instructions, such as those contained within the modules described herein. In their most basic configuration, these computing system(s) may each comprise at least one memory system and at least one physical processor.

The term “memory” or “memory system,” as used herein, generally represents any type or form of volatile or non-volatile storage system or medium capable of storing data and/or computer-readable instructions. In one example, a memory system may store, load, and/or maintain one or more of the modules described herein. Examples of memory systems comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory system. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing system, may cause the computing system to perform one or more tasks, such as the method step.

In addition, one or more of the systems described herein may transform data, physical systems, and/or representations of physical systems from one form to another. Additionally or alternatively, one or more of the modules recited herein may transform a processor, volatile memory, non-volatile memory, and/or any other portion of a physical computing system from one form of computing system to another form of computing system by executing on the computing system, storing data on the computing system, and/or otherwise interacting with the computing system.

The term “computer-readable medium,” as used herein, generally refers to any form of system, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following numbered clauses.

Clause 1. An OCT system for measuring reflectance of tissue layers, comprising: an array of VCSELs configured to generate an array of light beams and sweep a wavelength of each of the light beams; an interferometer coupled to the VCSEL array, the interferometer comprising a measurement optical path and a reference optical path to generate a plurality of interference signals; an array detector coupled to the interferometer to receive the plurality of interference signals; and a processor coupled to the array detector, the processor configured with instructions to determine the reflectance of the tissue layers in response to the plurality of interference signals.

Clause 2. The system of clause 1, wherein the processor is configured to generate a plurality of A-scans of the tissue layers at a plurality of locations, the plurality of locations corresponding to locations of the array of light beams imaged onto the tissue layers.

Clause 3. The system of any of clauses 1 to 2, wherein each of the plurality of A-scans corresponds a location of a VCSEL of the VCSEL array imaged onto the tissue layers.

Clause 4. The system of any of clauses 1 to 3, further comprising one or more lenses arranged to form a reference image of the array of VCSELs on a reference mirror and a measurement image of the array in the eye and combine the measurement image with the reference image to generate a plurality of interferograms.

Clause 5. The system of any of clauses 1 to 4, wherein reference image of the array comprises a plurality of reference beams at a plurality of locations on the reference mirror corresponding to locations of the plurality of VCSELs and wherein the measurement image comprises a plurality of measurement beams at a plurality of locations on the retina corresponding to the locations of the plurality of VCSELs and wherein the plurality of reference beams overlaps with the plurality of reference beams at a plurality of corresponding locations on the detector to generate a plurality of interferograms.

Clause 6. The system of any of clauses 1 to 5, wherein each of the plurality of interferograms corresponds to one of the plurality of VCSELs, one of the plurality of locations on the reference mirror and one of the plurality of locations on the retina.

Clause 7. The system of any of clauses 1 to 6, wherein the one or more lenses comprises an array of lenslets to generate a plurality of images of the measurement image and the reference image, each of the plurality of lenslets generating an image of the measurement image and the reference image.

Clause 8. The system of any of clauses 1 to 7, wherein the mirror comprises a deformable mirror and optionally wherein the deformable mirror comprises one or more of a deformable membrane mirror or a segmented mirror to compensate for an optical path difference between the tissue layers and the reference mirrors.

Clause 9. The system of any of clauses 1 to 8, wherein the detector array is configured to sample the plurality of interference signals at a plurality of locations at a rate of at least about 1000 Hz, the array of VCSELs comprises at least about 100 VCSELs and the array detector comprises at least about 10,000 pixels.

Clause 10. The system of any of clauses 1 to 9, further comprising one or more lenses configured to image the array of VCSELs inside the eye to generate the plurality of interference signals.

Clause 11. The system of any of clauses 1 to 10, wherein each VCSEL of the array is configured to vary the wavelength with one or more heating or an index change of a gain medium within said each VCSEL.

Clause 12. The system of any of clauses 1 to 11, wherein said each VCSEL of the array is configured to vary the wavelength without a MEMS mirror.

Clause 13. The system of any of clauses 1 to 12, wherein each VCSEL of the array is configured to sweep the wavelength by an amount within a range from about 5 nm to about 20 nm.

Clause 14. The system of any of clauses 1 to 13, further comprising a lenslet array to image the array of light beams returned from the retina.

Clause 15. The system of any of clauses 1 to 14, wherein each lens of the lenslet array forms an image of the array of light beams on the array detector.

Clause 16. The system of any of clauses 1 to 15, wherein a number of lenses of the lenslet array corresponds to a multiplier of a number of images of the array of light beams onto the array detector and optionally wherein number of elements of the VCSEL array imaged onto the detector is multiplied by the number of lenslets.

Clause 17. The system of any of clauses 1 to 16, wherein each lens of the lenslet comprises a phase matched with phases of other lenslets of the array.

Clause 18. The system of any of clauses 1 to 17, wherein the OCT system does not comprise a scanning mirror to scan the array of light beams laterally along the tissue layers.

Clause 19. The system of any of clauses 1 to 18, wherein the OCT system comprises a scanning mirror to scan the array of light beams laterally along the tissue layers.

Clause 20. A method of measuring reflectance of tissue layers with OCT, comprising: generating an array of light beams from a plurality of light sources; sweeping a wavelength of each of the plurality of light sources; generating a plurality of interference signals with an interferometer coupled to the array of light beams, the interferometer comprising a measurement optical path and a reference optical path; receiving the plurality of interference signals with an array detector coupled to the interferometer; and determining, with a processor, the reflectance of the tissue layers in response to the plurality of interference signals.

Clause 21. An OCT system for measuring reflectance of tissue layers, comprising: a plurality of light sources configured to generate an array of light beams and sweep a wavelength of each of the light beams; an interferometer coupled to the array of light beams, the interferometer comprising a measurement optical path and a reference optical path to generate a plurality of interference signals; an array detector coupled to the interferometer to receive the plurality of interference signals; and a processor coupled to the detector, the processor configured with instructions to determine the reflectance of the tissue layers in response to the plurality of interference signals.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. An OCT system for measuring reflectance of tissue layers, comprising:
an array of VCSELs configured to generate an array of light beams and sweep a wavelength of each of the light beams;
an interferometer coupled to the VCSEL array, the interferometer comprising a measurement optical path and a reference optical path to generate a plurality of interference signals;
an array detector coupled to the interferometer to receive the plurality of interference signals; and
a processor coupled to the array detector, the processor configured with instructions to determine the reflectance of the tissue layers in response to the plurality of interference signals.

2. The OCT system of claim 1, wherein the processor is configured to generate a plurality of A-scans of the tissue layers at a plurality of locations, the plurality of locations corresponding to locations of the array of light beams imaged onto the tissue layers.

3. The OCT system of claim 2, wherein each of the plurality of A-scans corresponds a location of a VCSEL of the VCSEL array imaged onto the tissue layers.

4. The OCT system of claim 1, further comprising one or more lenses arranged to form a reference image of the array of VCSELs on a reference mirror and a measurement image of the array in the eye and combine the measurement image with the reference image to generate a plurality of interferograms.

5. The OCT system of claim 4, wherein reference image of the array comprises a plurality of reference beams at a plurality of locations on the reference mirror corresponding to locations of the plurality of VCSELs and wherein the measurement image comprises a plurality of measurement beams at a plurality of locations on the retina corresponding to the locations of the plurality of VCSELs and wherein the plurality of reference beams overlaps with the plurality of reference beams at a plurality of corresponding locations on the detector to generate a plurality of interferograms.

6. The OCT system of claim 5, wherein each of the plurality of interferograms corresponds to one of the plurality of VCSELs, one of the plurality of locations on the reference mirror and one of the plurality of locations on the retina.

7. The OCT system of claim 4, wherein the one or more lenses comprises an array of lenslets to generate a plurality of images of the measurement image and the reference image, each of the plurality of lenslets generating an image of the measurement image and the reference image.

8. The OCT system of claim 4, wherein the mirror comprises a deformable mirror and optionally wherein the deformable mirror comprises one or more of a deformable membrane mirror or a segmented mirror to compensate for an optical path difference between the tissue layers and the reference mirrors.

9. The OCT system of claim 1, wherein the detector array is configured to sample the plurality of interference signals at a plurality of locations at a rate of at least about 1000 Hz, the array of VCSELs comprises at least about 100 VCSELs and the array detector comprises at least about 10,000 pixels.

10. The OCT system of claim 1, further comprising one or more lenses configured to image the array of VCSELs inside the eye to generate the plurality of interference signals.

11. The OCT system of claim 1, wherein each VCSEL of the array is configured to vary the wavelength with one or more heating or an index change of a gain medium within said each VCSEL.

12. The OCT system of claim 11, wherein said each VCSEL of the array is configured to vary the wavelength without a MEMS mirror.

13. The OCT system of claim 1, wherein each VCSEL of the array is configured to sweep the wavelength by an amount within a range from about 5 nm to about 20 nm.

14. The OCT system of claim 1, further comprising a lenslet array to image the array of light beams returned from the retina.

15. The OCT system of claim 14, wherein each lens of the lenslet array forms an image of the array of light beams on the array detector.

16. The OCT system of claim 14, wherein a number of lenses of the lenslet array corresponds to a multiplier of a number of images of the array of light beams onto the array detector and optionally wherein number of elements of the VCSEL array imaged onto the detector is multiplied by the number of lenslets.

17. The OCT system of claim 14, wherein each lens of the lenslet comprises a phase matched with phases of other lenslets of the array.

18. The OCT system of claim 1, wherein the OCT system does not comprise a scanning mirror to scan the array of light beams laterally along the tissue layers.

19. The OCT system of claim 1, wherein the OCT system comprises a scanning mirror to scan the array of light beams laterally along the tissue layers.

20. (canceled)

21. An OCT system for measuring reflectance of tissue layers, comprising:
a plurality of light sources configured to generate an array of light beams and sweep a wavelength of each of the light beams;
an interferometer coupled to the array of light beams, the interferometer comprising a measurement optical path and a reference optical path to generate a plurality of interference signals;
an array detector coupled to the interferometer to receive the plurality of interference signals; and
a processor coupled to the detector, the processor configured with instructions to determine the reflectance of the tissue layers in response to the plurality of interference signals.
